(19) United States
(12) Patent Application Publication
(10) Pub. No.: US 2013/0052730 A1
(43) Pub. Date: Feb. 28, 2013

(54) METHODS AND SYSTEM FOR CRYOGENIC PRESERVATION OF CELLS

(71) Applicant: Brian Schryver, Redwood City, CA (US)

(72) Inventor: Brian Schryver, Redwood City, CA (US)

(73) Assignee: BIOCISION, LLC, Mill Valley, CA (US)

(21) Appl. No.: 13/592,702

(22) Filed: Aug. 23, 2012

Related U.S. Application Data
(60) Provisional application No. 61/527,649, filed on Aug. 26, 2011.
(60) Provisional application No. 61/602,444, filed on Feb. 23, 2012.

(51) Int. Cl. C12M 1/02; C12N 5/00

(52) U.S. Cl. 435/374; 435/307.1

(57) ABSTRACT

Methods and systems for cryogenic preservation of cells.

### RELATED APPLICATIONS

This application claims priority to United States Provisional Patent Application Ser. Nos. 61/527,649, filed on Aug. 26, 2011, and 61/602,444, filed on Feb. 23, 2012, both entitled METHODS AND SYSTEM FOR CRYOGENIC PRESERVATION OF CELLS, which are incorporated herein by reference in its entirety.

### BACKGROUND OF THE INVENTION

Cryogenic preservation of cells in suspension is a well established and accepted technique for long term archival storage and recovery of live cells. As a general method, cells are suspended in a cryopreservation media typically consisting of salt solutions, buffers, nutrients, growth factors, proteins, and cryopreservatives. The cells are then distributed to archival storage containers of the desired size and volume, and the containers are then reduced in temperature until the container contents are frozen. Typical long-term archival conditions include liquid nitrogen vapor storage where temperatures are approximately −190 degrees Celsius.

The recovery of live cells preserved by such methods is dependent upon minimizing injurious ice crystal growth in the intracellular region during both the freezing and thawing processes. A combination of two methods for reducing intracellular ice crystal growth is typically practiced in the freezing process. The first method involves adding a cryoprotectant compound to the tissues or cell suspension solution. The cryoprotectant permeates the cell membrane and inhibits ice crystal nucleation and growth both extracellularly and intracellularly. The second method involves managing the reduction in sample temperature over time.

As ice forms in the extracellular fluid, the solute salt and buffer components concentrate in the remaining liquid phase. The concentrated solutes impose an osmotic gradient upon the cell membrane that draws water from the intracellular region. If the freezing of the intracellular solution is coincident with the appropriate level of water content, the size of the crystals resulting from the crystallization of the remaining intracellular water will not be of sufficient magnitude to damage the cell. If, however, the degree of water removal from the cell is excessive, or if the exposure of the cells to concentrated extracellular solutes is too long in duration, damage to cellular structures will incur, resulting in reduced cell recovery upon thawing.

There is a range of intracellular water content appropriate for cell survival during freezing. Ideally, ensuring that the intracellular solidification coincides with the correct intracellular water content can be accomplished by controlling the temperature reduction rate profile of the sample. The appropriate temperature reduction profile is dependent upon multiple factors such as cell membrane permeability, cell size and concentration of solutes and cryoprotectant components, so establishing the optimal reduction profile can be difficult. However, once the appropriate reduction profile is established for a specific cell type, the survival rate upon thawing could be consistently reproduced by applying the same optimal temperature reduction profile to all samples of the given cell type.

Cryopreservation techniques similar to those describe above have been applied to cell suspensions. As a significant percentage of cells are cultured as adherent populations, gentle removal of the cells from the culture surface is required prior to suspension. This is typically accomplished through the brief application of proteolytic enzyme solutions to the cell culture, which sever the adhesive proteins by which the cells anchor themselves to the culture surface. Following enzymatic treatment, the cells, now in free suspension, will typically undergo an exchange of the growth medium for a cryopreservation medium in which the cell suspension is to be frozen. The cell suspension in the cryopreservation medium is then typically dispensed in smaller volumes to vials that are designed to withstand cryogenic temperatures. The vials are then frozen at rate of temperature decline intended to optimize the survival of the cells. As the need arises to recover the cell culture, a vial sample is retrieved from cryogenic storage and thawed, after which the cells are transferred to growth media for recovery and expansion of the culture.

Volumes in the range of 0.25 ml to 5 ml are typically used for cryopreservation aliquots with cell concentrations of one to ten million cells per ml. However, significant benefits could be realized if viable cells could be recovered from much smaller volumes. For example, there remains a need for methods and devices that would enable cryogenically preserved cells stored in microplate arrays to be recovered for subsequent use in procedures such as cell based assays and other assays that do not require the larger numbers of cells typically used to reestablish a cell culture.

### BRIEF SUMMARY OF THE INVENTION

The various embodiments of the present invention meet the above-described needs. For example, in some embodiments the invention provides a kit that provides reagents for an assay, including the cells used in the assay in a ready-made frozen microplate format. Such kits allow the end-user to bypass the time-intensive and tedious steps of cell culture expansion and sub-plating to a microplate format before beginning an assay. Microplates are supplied in an industry standard footprint with well numbers typically ranging from 6 to 96 to 384 or more wells per plate.

In addition to the convenience features of storing frozen cell suspensions in a microplate format, the present invention provides methods and devices applicable to adherent cells, which now can also be stored frozen in a microplate format. Freezing adherent cells bypasses the steps of dislodging cells from a growth surface and preparation of a cell suspension prior to freezing. In addition, directly preserving adherent cell cultures provides benefits such as preservation of the extracellular matrix which cells develop during growth, and decreasing the recovery time, as cryopreserved suspended cells have to reestablish adhesion and normal cell function. As an example of the utility of the present method for preserving adherent cells, cells frozen in this manner allows for assay kits of the invention in which the cells are supplied as frozen preserved adherent cells that can be used shortly after thawing, or after a reduced time of cell recovery, as compared to cryopreserved, suspended cells.

As cryopreservation of cells includes a freezing step, which involves a controlled rate of temperature reduction, freezing cells in microplate format presents technical challenges. As a result of the two-dimensional array format of the wells on the microplate, during the temperature reduction of the freezing process, wells that are on the periphery of the array are exposed on one or more sides, while the more interior wells are surrounded by other wells. The centermost wells in the array are surrounded by multiple rows of wells, and due to the thermal mass and insulating aspects of the surrounding well and sample material, thermal energy encounters greater resistance to flow to the environment. This increased resistance imposes a reduced rate of temperature reduction for the inner wells as compared to the outer wells of the microplate. As the optimal recovery and viability of the cryogenically preserved cells is dependent upon the rate of temperature reduction during the freezing process, it is to be expected that a gradient of viability will be observed across the microplate unless a technique is applied that equalizes the rate of thermal energy reduction across all of the microplate wells. The devices of this invention solve this problem and provide temperature reduction uniformity in the wells of a microplate during the freezing process.

The devices of this invention comprise a material with greater thermal conductivity than the plastic material from which microplates are constructed. By placing the thermally conductive material in the form of a backing plate in direct contact with the underside of the microplate wells during the freezing process, thermal energy that would otherwise be transferred from the centermost wells through the microplate to the periphery of the plate is more readily conducted to the environment through the thin plastic of the bottom of the well to the highly conductive plate beneath the well. As the backing plate is constructed from a highly thermoconductive material, any temperature differential across horizontal planes through backing plate will be extremely small, as the distribution of thermal energy will rapidly equilibrate throughout the material. As all wells of the microplate are in direct contact with the backing plate, the rate of thermal energy transfer from the wells is uniform and, as a result, the temperature reduction rate and freezing rate is consistent across the wells of the microplate.

### BRIEF DESCRIPTION OF THE SEVERAL VIEWS OF THE DRAWINGS

In order that the manner in which the above-recited and other features and advantages of the invention are obtained will be readily understood, a more particular description of the invention briefly described above will be rendered by reference to specific embodiments thereof which are illustrated in the appended drawings. These drawings depict only typical embodiments of the invention and are not therefore to be considered to limit the scope of the invention.

FIG. 1 is an exploded perspective view of a passive device for freezing microplates in accordance with a representative embodiment of the present invention.

FIG. 2 is a cross section view of a passive device for freezing microplates in accordance with a representative embodiment of the present invention.

FIG. 3 is a graphic plot showing the freezing profile of samples in a microplate in accordance with a representative embodiment of the present invention.

FIG. 4 is a graphic plot showing the freezing profile of samples in a microplate in accordance with a representative embodiment of the present invention.

### DETAILED DESCRIPTION OF THE INVENTION

The presently preferred embodiments of the present invention will be best understood by reference to the drawings, wherein like reference numbers indicate identical or functionally similar elements. It will be readily understood that the components of the present invention, as generally described and illustrated in the Figures herein, could be arranged and designed in a wide variety of different configurations. Thus, the following more detailed description, as represented in the Figures, is not intended to limit the scope of the invention as claimed, but is merely representative of presently preferred embodiments of the invention.

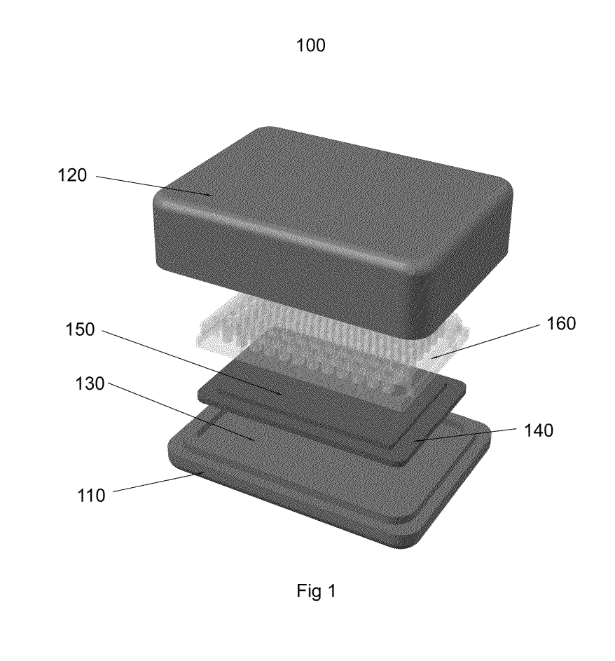

One embodiment of the invention is shown in exploded format in FIG. 1. In this Figure, an exploded view of a passive device for freezing microplates is shown. The Figure shows a base 110 that is constructed from an insulating material such as polyethylene foam, urethane foam, or styrene foam. In the base, a recess 130 is provided to receive and support a backing plate 140 that is typically constructed from a material with a thermal conductivity in the range of 150 watts per meter degree Kelvin to 430 watts per meter degree Kelvin, such as aluminum, aluminum alloy, copper, copper alloy, silver, or silver alloy or laminated layers of the same or similar materials. The backing plate comprises a raised stage with sufficient height such that the stage surface 150 is in direct contact with the underside of the 96 well flat-bottom microplate 160 and is the exclusive means of support for the microplate. The stage length and width are sufficient to provide contact with the entire undersurface of each of the wells of the microplate. An upper cover 120, constructed from an insulating material such as polyethylene foam, urethane foam or styrene foam, joins with base 110, to form a sealed chamber that contains the backing plate and microplate.

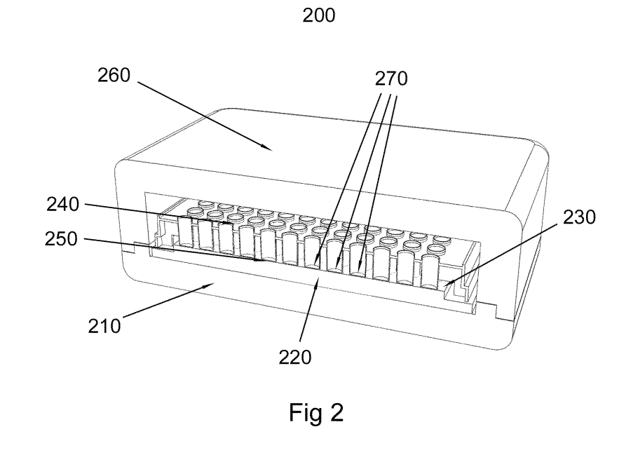

The assembly and function of the invention embodiment described in FIG. 1 is demonstrated in the cross-section illustration of FIG. 2. Suspended cell solutions are dispensed into the wells and adherent cells attach to the bottom surface 270 of the wells of the microplate 240. Prior to freezing, the growth medium is replaced with a reduced volume, typically in the range of 30 microliters to 150 microliters, of freezing medium. Alternatively, cell suspensions in freezing media can be dispensed into the microplate wells to be frozen as a cell suspension. The backing plate 220 is then placed into the receiving cavity of the insulating base 210, after which the microplate is placed directly on the stage surface 230 of the backing plate such that the underside of all of the wells forms a direct contact interface 250 with the backing plate stage surface. The microplate and backing stage are then enclosed in insulating material by placing the insulating cover 260 over the microplate, thereby forming a sealed chamber by mating with the base 210. The complete assembly 200 is then transferred to a cold environment, typically a mechanical freezer in the range of −70 to −80 degrees Celsius.

As heat from the assembly 200 is lost to the cold environment, the interior chamber temperature of the invention is reduced, resulting in a flow of thermal energy from the microplate, from the microplate well contents, and from the backing plate. The rate at which thermal energy is removed from the assembly depends upon the thickness of the insulation container, and the rate of temperature reduction is a function of the initial heat content within the chamber and the rate of thermal energy transfer to the environment. In the embodiment of the invention shown, the thermal energy contained within the liquid and cells in the microplate wells is conducted primarily through plastic bottom of the well to the more thermally conductive packing plate, exiting the device through the insulation material of the base. As the thermal conductivity of the backing plate is significantly greater than either the microplate plastic or the base insulation, the thermal energy rapidly equilibrates within the backing plate, resulting in the establishment of a very uniform temperature gradient between the backing plate and all wells of the microplate. As thermal energy flows along a temperature gradient, and as all conductive pathways from the microplate to the backing plate are identical, a uniform transfer of thermal energy occurs for all wells of the microplate.

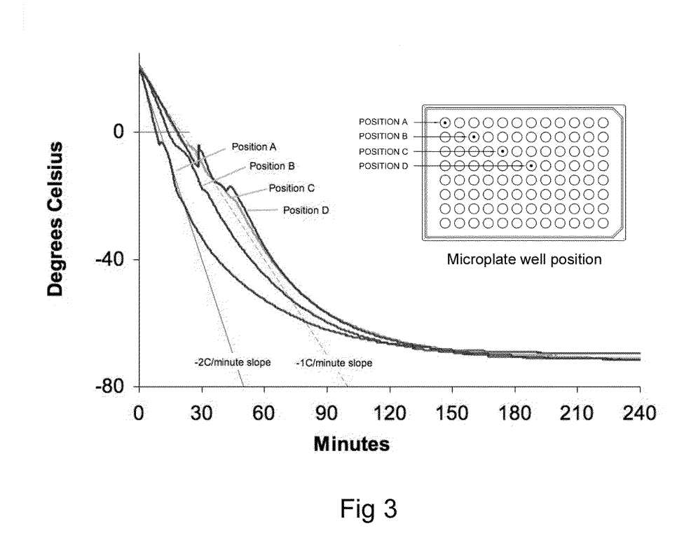

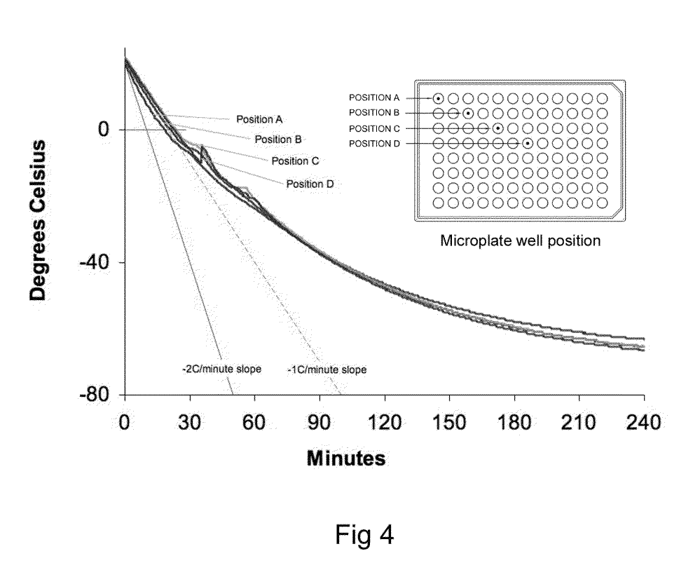

The effectiveness of the backing plate in increasing the uniformity of the temperature reduction rates and freezing rates of the well contents is illustrated in FIGS. 3 and 4. The graphic plots of FIG. 3 were generated using the device described in FIGS. 1 and 2, wherein each well of the 96-well microplate contained 50 microliters of a typical cell freezing medium consisting of 70 percent mammalian cell culture growth medium, 20 percent fetal calf serum, and 10 percent dimethylsulfoxide. A thermocouple probe was placed into each of 4 wells representing the outermost to the innermost wells of the array as shown in the diagram insert in FIG. 3. The thermocouple ends were held in position with the bead of the thermocouple in the center of the liquid using a plastic adaptor plug. The plate was covered with a plastic lid provided with the microplate by the manufacturer (Nunc) that was modified with access ports through which the thermocouple leads could pass. Additional access ports were introduced into the foam lid of the insulation encasement through which the thermocouple leads could be introduced. The backing plate was removed from the assembly for the purpose of determining the freezing rates of the monitored wells in the absence of the backing plate. The microplate was then placed directly onto the foam base. All profiles in FIG. 3 were generated simultaneously during one freezing of the plate. The traces of the temperature with time show a faster freezing rate with an initial slope of greater than −2 degrees Celsius per minute for the well at position A, while the slowest initial rates of approximately −1 degree per minute were observed in the innermost wells at positions C and D, with an intermediate rate observed for well B. The result indicates that, in the absence of a backing plate, the thermal energy flow from the central wells is restricted when compared to the wells at the plate periphery.

FIG. 4 displays the temperature as a function of time for the same device, microplate, and well contents used in FIG. 3, with the addition of the thermally conductive backing plate. When introduced into the same cold environment, the temperature profiles produced are significantly more uniform as compared to those in FIG. 3, indicating that the backing plate is effective in maintaining a consistent distribution and flow of thermal energy across the microplate.

As the rate of temperature reduction has a known effect upon the viability of a cryogenically preserved cell population upon thawing, it may be expected that cells dispensed to or cultured on a multi-well microplate and subsequently frozen under conditions where the temperature reduction profiles of the wells are non-uniform may contain regions of decreased viability upon thawing of the plate. The devices of this invention provide uniform freezing profiles across the microplate.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

What is claimed is:

1) A device wherein a thermally conductive backing plate is placed in contact with the underside of a microplate providing increased uniformity in the temperature reduction rate and freezing rate of the contents of all of the wells of the microplate.

2) The device of claim 1 wherein the thermally conductive backing plate is constructed from aluminum, aluminum alloys, copper, copper alloys, silver, silver alloys or similarly conductive materials.

3) The device of claim 1 wherein the backing plate and microplate are enclosed in an insulating material.

4) The device of claim 3, wherein the insulating material is a synthetic foam material such as polyethylene foam, urethane foam, or styrene foam.

5) The device of claim 1 wherein the temperature reduction process consists of placing the device into a cold environment.

6) The device of claim 1 wherein the backing plate comprises a plurality of stages for the purpose of providing a uniform temperature reduction rate and freezing rate to multiple microplates.

7) The device of claim 1 wherein the backing plate is cooled by a regulated mechanical or electronic refrigeration device, including but not limited to a thermoelectric cooler, or by regulated contact with low temperature gas, liquid, or solid phase-change material, including but not limited to solid carbon dioxide.

8) The device of claim 1 wherein the microplate wells contain cells that are adherent to an interior surface of the wells.

9) The device of claim 1 wherein the microplate wells contain a cell suspension.

10) A device for freezing cells in a microplate format as described herein by FIGS. 1 and 2.

11) A method for cryopreservation of suspended or adherent cells in a multi-well microplate in which the undersurface of the wells of the microplate is placed in contact with a thermally conductive material to increase uniformity of well temperatures during the cryogenic freezing process.
